(19) United States
(12) Patent Application Publication
(10) Pub. No.: US 2026/0014254 A1
(43) Pub. Date: Jan. 15, 2026

(54) TREATMENT OF AUTOIMMUNE DISEASES WITH BCMA-TARGETING ENGINEERED IMMUNE CELLS

(71) Applicant: CARIBOU BIOSCIENCE, INC., Berkeley, CA (US)

(72) Inventors: Steven B. Kanner, Los Angeles, CA (US); George Kwong, San Jose, CA (US)

(73) Assignee: CARIBOU BIOSCIENCE, INC., Berkeley, CA (US)

(21) Appl. No.: 18/847,531

(22) PCT Filed: Apr. 30, 2024
(86) PCT No.: PCT/US2024/026972

§ 371 Date: Sep. 16, 2024

Related U.S. Application Data
(60) Provisional application No. 63/499,398, filed on May 1, 2023.
(60) Provisional application No. 63/584,744, filed on Sep. 22, 2023.

(51) Int. Cl. A61K 40/31; A61K 31/52; A61K 31/675; A61K 31/7076; A61K 35/17; A61K 40/11; A61K 40/50; A61P 37/00; C07K 16/28; C12N 5/0783; C12N 9/22; C12N 15/113; C12N 15/86

(52) U.S. Cl. CPC A61K 40/31; A61K 31/52; A61K 31/675; A61K 31/7076; A61K 35/17; A61K 40/11; A61K 40/50; A61P 37/00; C07K 16/28; C12N 5/0636; C12N 9/226; C12N 15/113; C12N 15/86; A61K 2239/31; A61K 2239/38; C07K 2317/622; C07K 2319/03; C12N 2310/20

(57) ABSTRACT

The invention comprises methods and compositions for treating autoimmune diseases with BCMA-targeting engineered immune cells including T cells and natural killer (NK) cells. The engineered immune cells comprise an anti-BCMA chimeric antigen receptor (CAR). Methods of making the engineered cells, methods of administration, and treatment regimens are also disclosed.

### CROSS-REFERENCE TO RELATED APPLICATIONS

This application claims priority to the U.S. provisional application Ser. No. 63/499,398 filed on May 1, 2023 and to the U.S. provisional application Ser. No. 63/584,744 filed on Sep. 22, 2023.

### FIELD OF THE INVENTION

The invention relates to therapies utilizing engineered cells expressing a chimeric antigen receptor (CAR-T cells and CAR-NK cells) targeting BCMA, and more specifically, to methods of using BCMA-targeting CAR-T cells and CAR-NK cells to treat autoimmune diseases.

### STATEMENT REGARDING FEDERALLY SPONSORED RESEARCH

None.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 22, 2024, is named “CBI050.30.xml” and is 2,404 bytes in size. The sequence listing contained in this .XML file is part of the specification and is hereby incorporated by reference herein in its entirety.

### BACKGROUND OF THE INVENTION

Lupus and rheumatoid arthritis are two of the most prevalent autoimmune diseases, affecting an estimated 5 million and 14 million people world-wide. Lupus (systemic lupus erythematosus, SLE) affects women of childbearing age. Rheumatoid arthritis (RA) strikes both genders between ages of 35 and 50 often resulting in disability. SLE and RA are autoimmune diseases for which no cure exists, and symptoms are often inadequately managed with medication. Autoimmune disease results from abnormal activity of the immune system including B and T cells directed against “self” or autoantigens. Current treatment includes high-dose corticosteroids to effect general immunosuppression.

Lupus is characterized by the presence of B cells with antibodies against cellular nucleoproteins. Therapies developed against B cell lymphomas (B cell depleting therapy) have been successfully used to manage lupus and multiple sclerosis. These therapies include monoclonal antibodies (mAbs) targeting CD19, CD20, B cell maturation antigen (BCMA), or BAFF-R. Unfortunately, mAb therapies usually require weekly intravenous administration with beneficial effect seen at six weeks after the primary infusion. For some patients, the symptoms return nine months post-infusion.

For example, Rituximab® (Rituxan®) is an anti-CD20 antibody targeting B cells. It has been shown to be effective against lupus. However, unlike with the treatment of tumors, management of autoimmune disease requires repeated administrations of the therapeutic agent and over time, resistance develops.

Multiple sclerosis (MS) is another immune system disease where the myelin sheath surrounding nerve fibers is systematically destroyed resulting in progressive disability. In 2020, a total of 2.8 million people were estimated to live with MS worldwide and the prevalence is rising (Walton, C., et al. (2020) Rising prevalence of multiple sclerosis worldwide: Insights from the Atlas of MS, third edition, Mult. Scler., 26(14)1816-1821). B cells play a central role in the pathogenesis of MS by activating and recruiting autoimmune T cells. Several B cell-targeting antibody therapies for MS are available. Alemtuzumab targets CD52 and was originally developed to treat chronic lymphocytic leukemia (CLL), a B cell malignancy. Ocrelizumab targets CD20 and was developed exclusively to treat MS. Rituximab and ofatumumab (both anti-CD20 antibodies) have been used off label in the treatment of MS. Ublituximab (also anti-CD20) and ofatumumab are undergoing clinical studies for approval in MS. Anti-CD19 CAR-T cell therapy has been shown to ameliorate experimental autoimmune encephalomyelitis, a mouse model for MS. Importantly, CAR-T cells were able to penetrate CNS and deplete B cells present in CNS. (Gupta, et al., (2023) CAR-T cell-mediated B cell depletion in central nervous system autoimmunity, Neurology Neuroimmunology and Neuroinflammation, 10:e200080.)

Anti-CD19 and anti-CD20 antibody therapies are associated with severe side effects resulting from immunosuppression, including progressive multifocal leukoencephalopathy (PML) and reactivation of Hepatitis B. There is a need for potent and safe therapy for lupus, rheumatoid arthritis and multiple sclerosis that would be well tolerated by patients.

### SUMMARY OF THE INVENTION

In one embodiment, the invention is a method of treating an autoimmune disease in a patient, the method comprising: administering to the patient an amount of a composition comprising BCMA-targeting engineered immune cells, thereby improving one or more symptoms of the autoimmune disease in the patient. In some embodiments, the autoimmune disease is selected from a group consisting of: Systemic Lupus Erythematosus (SLE), Rheumatoid Arthritis (RA), Type 1 Diabetes (T1D), Sjögren's syndrome, Neuromyelitis optica syndrome disorders (NMOSD), Myasthenia Gravis, Ankylosing spondylitis, Pemphigus vulgaris (PV), and Multiple Sclerosis (MS). In some embodiments, the patient is a human. In some embodiments, the one or more symptoms of the autoimmune disease is selected from the group consisting of proteinuria, alopecia, increased IgM and IgG antibody titers, the presence of anti-nucleoprotein IgG or IgM in blood serum, increased B cell counts in blood plasma, complement C3 and C5 levels in blood serum, and the presence of skin lesions or discoloration.

In some embodiments, the antibody-producing cells are B cells. In some embodiments, the BCMA-targeting engineered immune cells are CAR-T cells expressing an anti-BCMA chimeric antigen receptor (CAR). In some embodiments, the BCMA-targeting engineered immune cells are CAR-natural killer (NK) cells expressing an anti-BCMA chimeric antigen receptor (CAR). In some embodiments, cells are allogeneic. In some embodiments, the anti-BCMA CAR comprises an anti-BCMA scFv, a transmembrane domain and an intracellular stimulatory domain. In some embodiments, the anti-BCMA CAR further comprises a signal peptide and a hinge. In some embodiments, the anti-BCMA CAR comprises scFv consisting of SEQ ID NO: 1, a CD8 hinge, a CD8 transmembrane domain, a 4-1BB co-stimulatory domain and a CD3 zeta signaling domain.

In some embodiments, the anti-BCMA CAR is encoded by a nucleic acid comprising a coding sequence for the anti-BCMA CAR and a promoter. In some embodiments, the nucleic acid is integrated into the genome of the engineered immune cell. In some embodiments, the integration of the nucleic acid coding for the anti-BCMA CAR is performed using a CRISPR nuclease and a nucleic acid-targeting nucleic acid (NATNA). In some embodiments, prior to the integration, the nucleic acid coding for the anti-BCMA CAR is delivered into the immune cell via a viral vector.

In some embodiments, the amount of the composition administered to the patient comprises a dose of the BCMA-targeting engineered immune cells equivalent to 1/1000 of the dose used to treat B-cell malignancies with the same BCMA-targeting engineered immune cells. In some embodiments, the amount of the composition administered to the patient comprises between 10,000 and 100,000,000 of the BCMA-targeting engineered immune cells. In some embodiments, the amount of the composition administered to the patient comprises between 100 and 1,000,000 of the BCMA-targeting engineered immune cells per kilogram of body weight of the patient. In some embodiments, the amount of the composition administered to the patient comprises about 50,000 of the BCMA-targeting engineered immune cells. In some embodiments, the amount of the composition administered to the patient comprises about 800 of the BCMA-targeting engineered immune cells per kilogram of body weight of the patient. In some embodiments, the amount of the composition administered to the patient comprises fewer than 50,000,000 and no fewer than 50,000 of the BCMA-targeting engineered immune cells. In some embodiments, the amount of the composition administered to the patient comprises fewer than 800,000 and no fewer than 800 of the BCMA-targeting engineered immune cells per kilogram of body weight of the patient.

In some embodiments, the administering is performed intravenously. In some embodiments, the administering is performed 2-4 times per year. In some embodiments, prior to the administering, the patient undergoes lymphodepletion. In some embodiments, the lymphodepletion comprises administration of a compound selected from a group consisting of cyclophosphamide, fludarabine, azathioprine, methotrexate, mycophenolate, a calcineurin inhibitor, and volcosporin. In some embodiments, the lymphodepletion comprises administering cyclophosphamide at 300 mg/m² per day for up to 3 days. In some embodiments, the lymphodepletion further comprises administering fludarabine at 30 mg/m² per day for up to 3 days. In some embodiments, the method further comprises assessing the patient for improvements in one or more symptoms selected from the group consisting of proteinuria, alopecia, increased IgM and IgG antibody titers, the presence of anti-nucleoprotein IgG or IgM in blood serum, complement C3 and C5 levels in blood serum, increased B cell counts in blood plasma, and the presence of skin lesions or discoloration. In some embodiments, method further comprises increasing the dose of the BCMA-targeting engineered immune cells administered to the patient if an improvement is not observed.

In some embodiments, the composition further comprises one or more pharmaceutically acceptable excipients. In some embodiments, the one or more excipients are selected from the group consisting of carbohydrates, inorganic salts, antimicrobial agents, antioxidants, surfactants, buffers, acids, bases, and combinations thereof. In some embodiments, the composition further comprises a freezing agent.

In one embodiment, the invention is a composition for treating an autoimmune disease comprising BCMA-targeting engineered immune cells in the amount equivalent to 1/1000 of s dose used to treat B-cell malignancies with the same BCMA-targeting engineered immune cells. In some embodiments, the autoimmune disease is selected from a group consisting of: Systemic Lupus Erythematosus (SLE), Rheumatoid Arthritis (RA), Type 1 Diabetes (T1D), Sjögren's syndrome, Pemphigus vulgaris (PV), and Multiple Sclerosis (MS). In some embodiments, the BCMA-targeting engineered immune cells are CAR-T cells expressing an anti-BCMA chimeric antigen receptor (CAR). In some embodiments, the BCMA-targeting engineered immune cells are CAR-natural killer (NK) cells expressing an anti-BCMA chimeric antigen receptor (CAR). In some embodiments, the cells are allogeneic. In some embodiments, the anti-BCMA CAR comprises an anti-BCMA scFv, a transmembrane domain and an intracellular stimulatory domain. In some embodiments, the anti-BCMA CAR further comprises a signal peptide and a hinge. In some embodiments, the anti-BCMA CAR comprises an scFv consisting of SED ID NO: 1, a CD8 hinge, a CD8 transmembrane domain, a 4-1BB co-stimulatory domain and a CD3 zeta signaling domain. In some embodiments, the composition comprises between 10,000 and 100,000 of the BCMA-targeting engineered immune cells. In some embodiments, the amount of the composition administered to the patient comprises between 100 and 1,000 of the BCMA-targeting engineered immune cells per kilogram of body weight of the patient. In some embodiments, the amount of the composition administered to the patient comprises about 50,000 of the BCMA-targeting engineered immune cells. In some embodiments, the amount of the composition administered to the patient comprises about 800 of BCMA-targeting engineered immune cells per kilogram of body weight of the patient. In some embodiments, the amount of the composition administered to the patient comprises fewer than 50,000,000 of the BCMA-targeting engineered immune cells. In some embodiments, the amount of the composition administered to the patient comprises fewer than 80,000 of the BCMA-targeting engineered immune cells per kilogram of body weight of the patient. In some embodiments, the composition further comprises one or more pharmaceutically acceptable excipients. In some embodiments, the one or more excipients are selected from the group consisting of carbohydrates, inorganic salts, antimicrobial agents, antioxidants, surfactants, buffers, acids, bases, and combinations thereof. In some embodiments, the composition further comprises a freezing agent.

### BRIEF DESCRIPTION OF THE DRAWINGS

FIG. 1 depicts an example of a nucleic acid expression construct encoding an anti-BCMA chimeric antigen receptor (CAR).

FIG. 2 depicts an example of an armoring mechanism that protects CAR-T cells from an attack by the patient's immune system.

FIG. 3 shows results of in vitro cytotoxicity assessment of anti-BCMA CAR-T cells against SLE-derived cellular fractions.

FIG. 4 shows measurements of total or autoimmune antibody concentrations in co-cultures of anti-BCMA CAR-T cells with SLE-derived cellular fractions.

### DETAILED DESCRIPTION OF THE INVENTION

### Definitions

Unless defined otherwise, technical, and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012). The following definitions are provided to aid in understanding of the disclosure. The term “therapeutic benefit” refers to an effect that improves the condition of the patient with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the tumor, or prevention of metastasis, or prolonging overall survival (OS) or progression free survival (PFS) of a patient with cancer.

The terms “pharmaceutically acceptable” and “pharmacologically acceptable” refer to molecular entities and compositions that do not produce an adverse, allergic, or other deleterious reaction in a patient. For example, the pharmaceutically and pharmacologically acceptable preparations should meet the standards set forth by the FDA Office of Biological Standards. The term “pharmaceutically acceptable carrier” and “excipient” refer to aqueous solvents (e.g., water, aqueous solutions of alcohols, saline solutions, sodium chloride, Ringer's solution, etc.), non-aqueous solvents (e.g., propylene glycol, polyethylene glycol, vegetable oil, and injectable organic esters), as well as dispersion media, coatings, surfactants, gels, antioxidants, preservatives (e.g., antibacterial or antifungal agents, anti-oxidants, chelating agents, and inert gases), isotonic agents, absorption delaying agents, stabilizers, binders, disintegration agents, lubricants, sweetening agents, flavoring agents, and dyes. The concentration and pH of the various components in a pharmaceutical composition are adjusted according to well-known parameters for each component.

The term “domain” refers to one region in a polypeptide which is folded into a particular structure independently of other regions.

The term “adoptive cell” refers to a cell that can be genetically modified for use in a cell therapy treatment. Examples of adoptive cells include macrophages, and lymphocytes including T cells and natural killer (NK) cells.

The term “cell therapy” refers to the treatment of a disease or disorder that utilizes genetically modified cells. The term “adoptive cell therapy (ACT)” refers to a therapy that uses genetically modified adoptive cells. Examples of ACT include T-cell therapies, CAR-T cell therapies, natural killer (NK) cell therapies and CAR-NK cell therapies.

The term “lymphocyte” refers to a leukocyte that is part of the vertebrate immune system. Lymphocytes include T-cells such as CD4⁺ and/or CD8⁺ cytotoxic T cells, alpha/beta T cells, gamma/delta T cells, and regulatory T cells. Lymphocytes also include natural killer (NK) cells, natural killer T (NKT) cells, cytokine induced killer (CIK) cells, and antigen presenting cells (APCs), such as dendritic cells. Lymphocytes also include tumor infiltrating lymphocytes (TILs).

The terms “effective amount” and “therapeutically effective amount” of a composition such as a cell therapy composition, refer to a sufficient amount of the composition to provide the desired response in the patient to whom the composition is administered. In the context of administering a combination of therapeutic compounds, the effective amount of each therapeutic compound in the combination may be different from the effective amount of each therapeutic compound administered alone.

The terms “peptide,” “polypeptide,” and “protein” are interchangeable and refer to polymers of amino acids, including natural and synthetic (unnatural) amino acids, as well as amino acids not found in naturally occurring proteins, e.g., peptidomimetics, and D optical isomers. A polypeptide may be branched or linear and be interrupted by non-amino acid residues. The terms also encompass amino acid polymers that have been modified through acetylation, disulfide bond formation, glycosylation, lipidation, phosphorylation, cross-linking, or conjugation (e.g., with a label). The polypeptide need not include the full-length amino acid sequence of the reference molecule but can include only so much of the reference molecule as necessary in order for the polypeptide to retain its desired activity. For example, polypeptides comprising full-length proteins, fragments thereof, polypeptides with amino acid deletions, additions, and substitutions are encompassed by the terms “protein” and “polypeptide,” as long as the desired activity is retained. For example, polypeptides with 95%, 90%, 80%, 70% or less of sequence identity with the reference polypeptide are included as long the desired activity is retained by the polypeptides. The determination of percent identity between two nucleotide or amino acid sequences may be accomplished using a mathematical algorithm such as BLAST, NBLAST and XBLAST described in Altschul, et al. (1990, J. Mol. Biol. 215:403-410) and available from the National Center for Biotechnology Information (NCBI).

The terms “CRISPR” (clustered regularly interspaced short palindromic repeats), “CRISPR-Cas” (CRISPR-associated protein) and “CRISPR system” refer to the genome editing tool derived from prokaryotic organisms and comprising a nucleic acid guide molecule and a sequence-specific nucleic acid-guided endonuclease capable of cleaving a target nucleic acid strand at a site complementary to a sequence in the nucleic acid guide.

The term “NATNA” (nucleic acid targeting nucleic acid) refers to a nucleic acid guide molecule of the CRISPR system. NATNA may be comprised two nucleic acid targeting polynucleotides (“dual guide”) including a CRISPR RNA (crRNA) and transactivating CRISPR RNA (tracrRNA). NATNA may be comprised a single nucleic acid targeting polynucleotide (“single guide”) comprising crRNA and tracrRNA connected by a fusion region (linker). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

B cells producing autoantibodies are at least one documented cause of autoimmune diseases such as lupus (SLE and other forms of lupus), rheumatoid arthritis (RA), Type 1 Diabetes (T1D), Sjögren's syndrome, and Multiple Sclerosis (MS).

A common characteristic of active B cells is surface expression of CD19, CD20, and CD52. Anti-CD19, anti-CD20, and anti-CD52 antibodies such as tafasitamab, rituximab, ofatumumab, alemtuzimab and others have been successfully used to treat B cell malignancies. In addition, CD19-targeting cytotoxic T cells including autologous and allogeneic CAR-T cells have been shown to effectively reduce the numbers of CD19-expressing malignant B cells in patients. Attempts to attack autoimmune B cells with CAR-T cells in a mouse model have been described in U.S. application Pub. No. US20180264038 Chimeric antigen receptor (CAR) T cells as therapeutic interventions for auto- and alloimmunity, U.S. application Pub. No. US2020078403 Use of chimeric antigen receptor modified cells to treat autoimmune disease, and U.S. application Pub. No. US20200085871 Methods of using cytotoxic T cells for treatment of autoimmune diseases.

More recently, attempts have been made to develop CAR-T therapies exclusively for autoimmune indications. The chimeric autoantibody receptors (CAARs) specifically target autoimmune B cell receptors (BCRs) on the surface of autoimmune B cells. In vitro proof-of-concept experiments have shown autoantigen-dependent cytotoxicity of CAAR-T cells against autoimmune B cells. See Ellebrecht, et al. (2016) Reengineering chimeric antigen receptor T cells for targeted therapy of autoimmune disease, Science 353:179-184, Zhang, et al., (2021) In vitro elimination of autoreactive B cells from rheumatoid arthritis patients by universal chimeric antigen receptor T cells, Ann Rheum Dis.; 80:176-184. While this approach promises precise elimination of autoimmune cells in each of the autoimmune diseases, it lacks universality of the more traditional B cell targets like CD19 and CD20.

However, existing anti-CD19 and anti-CD20 antibody therapies are associated with severe side effects resulting from immunosuppression. Treating autoimmune diseases requires prolonged or even life-long administration of therapies in order to alleviate a patient's symptoms while preserving the quality of life. For this reason, a therapeutic agent for autoimmune diseases must be effective in low doses and have minimal side effects.

B cell maturation antigen (BCMA) is an attractive target for treating autoimmune diseases. The CD19 and CD20 surface antigens are expressed throughout the lymphoid lineage. In contrast, BCMA is a surface antigen present only on mature B cells including germinal center B cells and much more robustly, on antibody-producing B cells, memory cells, and plasma cells, see Dogan, et al., (2020) B-cell maturation antigen expression across hematologic cancers: a systematic literature review, Blood cancer Journal, 10:73. For this reason, a BCMA-targeting agent spares the immature B cells of the bone marrow, while depleting the mature antibody-secreting (including autoantibody-secreting) cells. In some embodiments, a BCMA-targeting agent is used in a lower dose compared to a CD19 or CD20-targeting agent of similar design.

To further enable a low-dose administration, an engineered immune cell, including an autologous or an allogeneic anti-BCMA CAR-T cell or CAR-NK cell, may be armored or cloaked against the patient's immune system. Limiting the destruction of CAR-T or CAR-NK cells by the host's immune system ensures that a lower dose of the cells produces a therapeutic effect. In some embodiments, a BCMA-targeting cell armored against a patient's immune system is used in a lower dose compared to a BCMA-targeting cell of similar design that lacks the armoring modification.

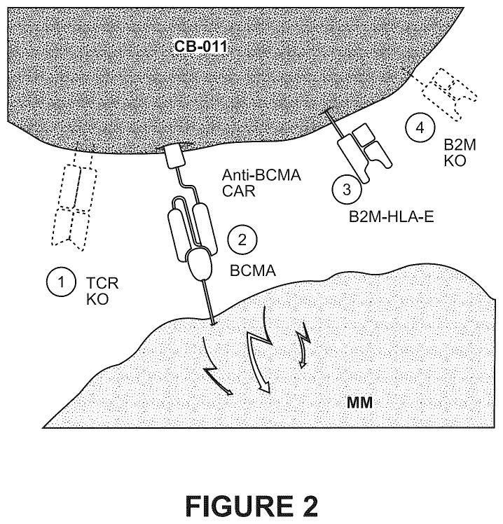

The autoimmune disease treatment disclosed herein comprises a low dose of well-tolerated anti-BCMA allogeneic CAR-T cells or CAR-NK cells that are armored against the patient's immune system by the disruption of the beta-2-microglobulin (B2M) gene and insertion of a B2M-HLA-E-peptide fusion into the B2M locus (FIG. 2).

In some embodiments, the invention comprises adoptive cells and the use of adoptive cells to treat or alleviate autoimmune diseases including lupus, rheumatoid arthritis, Type 1 Diabetes (T1D), Sjögren's syndrome, and Multiple Sclerosis (MS). Adoptive cells of the instant invention include lymphocytes, such as T cells, CAR-T cells, NK cells, iPSC-derived NK (iNK) cells, and CAR-NK cells.

In some embodiments, the invention utilizes T cells isolated from a healthy donor. In some embodiments, the T cells are obtained from a blood sample of a healthy donor via leukapheresis. Techniques for isolating lymphocytes are well known in the art, see, e.g., Smith, J. W. (1997) Apheresis techniques and cellular immunomodulation, Ther. Apher. 1:203-206. In some embodiments, the invention utilizes a T cell composition depleted of CD4+ T cells (T-helper cells) known to contribute to the symptoms of autoimmune disease. In some embodiments, the invention utilizes a T cell composition substantially free of CD4⁺ T cells.

In some embodiments, the invention utilizes natural killer (NK) cells isolated from a healthy donor, e.g., from peripheral blood mononuclear cells (PBMC), leukapheresis products (PBSC), bone marrow, or umbilical cord blood by methods well known in the art, see, e.g., Spanholtz, J. et al., (2011) Clinical-grade generation of active NK cells from cord blood hematopoietic progenitor cells for immunotherapy using a closed-system culture process, PloS one, 6(6), e20740, and Shah, N., et al., (2013) Antigen presenting cell-mediated expansion of human umbilical cord blood yields log-scale expansion of natural killer cells with anti-myeloma activity. PloS one, 8(10), e76781.

In some embodiments, the invention utilizes NK cells obtained by differentiating human embryonic stem cells (hESCs) or induced pluripotency stem cells (iPSCs). NKs differentiated from iPSCs are referred to as iNK cells.

In some embodiments, the NK cells are heterologous and are haplotype-matched for the patient in one or more HLA locus, one or more KIR locus or both.

In some embodiments, the isolated NK cell composition is depleted of CD3⁺ cells. In some embodiments, the isolated NK cell composition is enriched for CD56⁺ cells. In some embodiments, the isolated NK cell composition is enriched for CD45⁺ cells. In some embodiments, the isolated cell NK composition is enriched for CD56⁺/CD45⁺ cells. In some embodiments, a quality control measure or characterization step is applied to the isolated NK cell composition, e.g., determining the percentage of CD56⁺/CD3⁻, CD45⁺/CD3⁻ cells, CD56⁺/CD45⁺, or CD56⁺/CD45⁺/CD3⁻ in the composition. In some embodiments, the invention utilizes an NK cell composition substantially free of CD3⁺ cells.

In some embodiments, isolated lymphocytes are characterized in terms of specificity, frequency of each subtype, and function. In some embodiments, the isolated lymphocyte population is enriched for specific subsets of T cells, such as CD8⁺, CD25⁺, or CD62L⁺. See, e.g., Wang et al., Mol. Therapy—Oncolytics (2016) 3:16015. In some embodiments, the isolated NK cell composition is enriched for CD56⁺/CD45⁺ cells.

In some embodiments, the quality control measure or characterization step is applied to the cell-containing composition. In some embodiments, the quality control measure or characterization step is determining the percentage of CD56⁺/CD45⁺ cells in the composition by flow cytometry.

In some embodiments, after isolation, lymphocytes are activated in order to promote proliferation and differentiation into specialized lymphocytes. For example, T cells can be activated using soluble CD3/28 activators, or magnetic beads coated with anti-CD3/anti-CD28 monoclonal antibodies.

In some embodiments, the invention is a method of treating an autoimmune disease in a patient comprising administering to the patient a composition comprising immune cells expressing a BCMA-targeting protein. In some embodiments, the immune cell is selected from a T cell, a natural killer (NK) cell, an iNK cell. In some embodiments, the immune cell is selected from a CAR-T cell, a CAR-NK cell.

In some embodiments, the BCMA-targeting protein is an anti-BCMA T cell receptor. In some embodiments, the anti-BCMA T cell receptor in a chimeric antigen receptor (CAR). In some embodiments, the immune cells are CAR-T cells or CAR-NK cells.

In some embodiments, the CAR comprises an extracellular domain comprising a BCMA-binding region, a transmembrane domain and one or more intracellular co-activation (co-stimulatory) and activation (stimulatory) domains.

In some embodiments, the BCMA-binding region of the CAR is derived from a monoclonal antibody. In some embodiments, the BCMA-binding region comprises a fragment of the variable portion of the heavy chain (V_{H}) or a fragment of the variable portion of the light chain (V_{L}) of a single-chain variable fragment (scFv) or a camelid single domain antibody (V_{HH}). These fragments may be derived from a monoclonal antibody. The single-chain variable fragment (scFv) has the ability to bind BCMA. The scFv is comprised of the Fv regions of immunoglobulin heavy chain (H chain) and light chain (L chain) linked via a spacer sequence. In some embodiments, the BCMA-binding scFv, BCMA-targeting CAR-T cells and CAR-NK cells are the ones described in U.S. Pat. Nos. 10,927,182, 11,021,542, 11,142,583, and 11,299,549. In some embodiments, the BCMA-binding scFv is SEQ ID NO: 6 of the U.S. Pat. No. 10,927,182.

In some embodiments, the transmembrane domain of the CAR is derived from a membrane-bound or transmembrane protein. For example, the transmembrane domain of the CAR may be the transmembrane domain of a T cell receptor alpha-chain or beta-chain, a CD3-zeta chain, CD28, CD3-epsilon chain, CD2, CD45, CD4, CD5, CD8, CD9, CD16, CD22, CD33, CD37, CD64, CD80, CD86, CD134, CD137, ICOS, CD154, DNAM1, NKp44, NKp46, NKG2D, 2B4, or GITR. In some embodiments, the transmembrane domain of the CAR is the CD8 transmembrane domain. In some embodiments, the transmembrane domain of the CAR is the CD8A transmembrane domain.

The intracellular signaling domain of a CAR is responsible for activation of one or more effector functions of the immune cell expressing the CAR. In some embodiments, the intracellular signaling domain of the CAR comprises a part of or the entire sequence of the CD3-zeta chain, CD3-epsilon chain, CD2, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR4, TLR5, TLR6, MyD88, CD40 or a combination thereof. In some embodiments the intracellular domain of the CAR consists of 4-1BB and CD3 zeta chain.

In some embodiments, the CAR comprises a hinge domain. In some embodiments the hinge domain of the CAR is the CD8 hinge domain. In some embodiments the hinge domain of the CAR is the CD8A hinge domain.

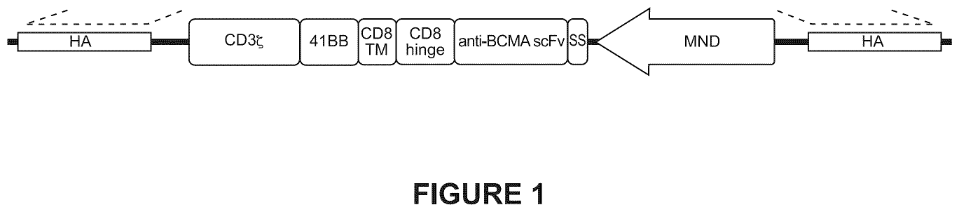

An exemplary anti-BCMA chimeric antigen receptor (CAR) construct is shown in FIG. 1. The CAR comprises a signal sequence (SS), an anti-BCMA scFv, the CD8 hinge domain, the CD8 transmembrane domain (TM), and the 4-1BB and the CD3-zeta intracellular domains. The expression is driven by the MND promoter. The construct is inserted into the cellular genome with the help of two flanking homology arms (HA).

In some embodiments, the CAR is a fully human protein or is humanized to reduce immunogenicity in human patients. In some embodiments, the nucleic acid sequence encoding the CAR is optimized for codon usage in human cells.

The nucleic acid encoding the CAR may be introduced into a cell as a genomic DNA sequence or a cDNA sequence. The cDNA sequence comprises the open reading frame for the translation of the CAR and in some embodiments, further comprises untranslated elements that improve for example, the stability or the rate of translation of the CAR mRNA.

In some embodiments, the cell used to treat autoimmune disease (T cell, a natural killer (NK) cell, an iNK cell, a CAR-T cell, or a CAR-NK cell) further comprises a genome modification resulting in armoring of the cell against an attack by the immune system of a recipient autoimmune disease patient. In some embodiments, the armoring modification comprises protection from recognition by the cytotoxic T cells of the host. Cytotoxic T cells recognize MHC Class I antigen. MHC Class I molecule is comprised of beta-2 microglobulin (B2M) associated with heavy chains of HLA-I proteins (selected from HLA-A, HLA-B, HLA-C, HLA-E, HLA-F and HLA-G) on the surface of the cell. The B2M/HLA-I complex on the surface of the allogeneic cell is recognized by cytotoxic CD8⁺ T cells and if HLA-I is recognized as non-self, the allogeneic cell is killed by the T cells. In some embodiments, the cells of the invention comprise an armoring genomic modification comprising a disruption of the B2M gene and therefore, disruption of the MHC Class I antigen recognition and cytotoxic T cell attack.

In some embodiments, the armoring genome modification comprises disruption of recognition by the NK cells of the host. NK cells recognize cells without MHC-I protein as “missing self” and kill such cells. NK cells are inhibited by HLA-I molecules, including HLA-E, a minimally polymorphic HLA-I protein. In some embodiments, the cells of the invention comprise a first armoring genomic modification comprising a disruption of the B2M gene and therefore, disruption of the MHC Class I antigen recognition and cytotoxic T cell attack, and further comprise a second armoring genomic modification comprising an insertion of an HLA-E gene fused to beta-2-microglobulin (B2M) gene, and therefore, expression of the HLA-E/B2M construct and armoring the cells from an attack by NK cells. See, e.g., Gornalusse et al., (2017) HLA-E-expressing pluripotent stem cells escape allogeneic responses and lysis by NK cells, Nat. Biotechnol. (2017) 35:765-772.

An example of armoring is shown in FIG. 2. The T cell receptor alpha constant (TRAC) gene was knocked out (KO) to prevent expression of the TCR alpha chain and assembly of cell-surface TCR α/β heterodimer complexes thereby reducing potential graft versus host disease (GvHD). An anti-BCMA CAR expression cassette was inserted into the TRAC locus. The beta-2-microglobulin (B2M) gene was knocked out to prevent cell-surface expression of major and minor histocompatibility complex (MHC) Class I antigens to reduce host T cell-mediated rejection. An expression cassette of a fusion protein combining B2M and human leukocyte antigen, Class 1, E (B2M-HLA-E-peptide) was inserted into the B2M locus to inhibit host NK cell-mediated rejection. (Garner, E., Degagne, E., et al., E. A BCMA-specific allogeneic CAR-T cell therapy (CB-011) genome engineered to express an HLA-E fusion transgene to prevent immune cell rejection, Poster LB009, American Association for Cancer Research (AACR) Annual Meeting, Apr. 10, 2022).

In some embodiments, the armoring modification comprises transcriptionally silencing or disrupting one or more immune checkpoint gene. In some embodiments, the one or more immune checkpoint gene is selected from PD1 (encoded by the PDCD1 gene), CTLA-4, LAG3, Tim3, BTLA, BY55, TIGIT, B7H5, LAIR1, SIGLEC10, and 2B4 as disclosed in the U.S. application publication US20150017136 Methods for engineering allogeneic and highly active T cell for immunotherapy.

In some embodiments, the patient receiving the treatment with immune cells expressing a BCMA-targeting protein is monitored to assess the clinical manifestations of the autoimmune disease. The symptoms are expected to diminish with treatment described herein. In some embodiments, the patient is assessed for clinical manifestations of the autoimmune disease prior to the administration of the immune cells expressing the BCMA-targeting protein. In some embodiments, the patient is assessed hourly, daily, weekly, or monthly after the first administration of the T cells or NK cells expressing the BCMA-targeting protein. In some embodiments, the patient is assessed in connection with a daily, weekly, or monthly regimen of administration of immune cells expressing the BCMA-targeting protein.

In some embodiments, the clinical manifestations of the autoimmune disease include one or more of proteinuria, alopecia, organ enlargement, the presence of hypercellular glomeruli, IgG tissue deposits, IgM and IgG antibody titers and IgG or IgM antinuclear antibody in blood serum, an increase in the total number or concentration of CD3⁺CD8⁺ cells in the blood plasma, an increase in the total number or concentration of B cells in the blood plasma, complement C3 levels, complement C4 levels, and the presence of skin lesions or discoloration. Accordingly, the patient is assessed for the clinical manifestations of the autoimmune disease by one or more of urine analysis, blood analysis (including total blood count), and physical evaluation.

In some embodiments, the total number or concentration of B cells in the blood plasma is assessed by flow cytometry. In some embodiments, the presence of the IgG or IgM antinuclear antibody in blood serum is assessed by ELISA.

In some embodiments, the patient is assessed for the presence and relative number of immune cells expressing the BCMA-targeting protein, such as T cells, NK cells, CAR-T cells, or CAR-NK cells. In some embodiments, the presence and relative number of the cells is assessed by one or more methods selected from flow cytometry, ELISA, fluorescent microscopy, fluorescent in situ hybridization (FISH), PCR and RT-PCR aimed at detecting the presence of the BCMA-targeting protein, the gene encoding the BCMA-targeting protein, or the mRNA encoding the BCMA-targeting protein respectively.

In some embodiments, the anti-BCMA CAR is encoded by a nucleic acid construct introduced into the cell used to treat autoimmune disease (T cell, a natural killer (NK) cell, or an iNK cell). In some embodiments, the anti-BCMA CAR expression construct comprises a coding sequence for the BCMA-targeting CAR, and a promoter.

In some embodiments, the BCMA-targeting CAR expression construct is introduced via an expression vector or an RNA encoding the BCMA-targeting CAR protein. In some embodiments, the target cells are contacted with the nucleic acid encoding the BCMA-targeting CAR in vitro, in vivo or ex vivo.

In some embodiments, the vector is a viral vector (e.g., a retroviral vector, adenoviral vector, adeno-associated viral vector, or lentiviral vector). Suitable vectors are non-replicating in the target cells. In some embodiments, the vector is selected from or designed based on SV40, EBV, HSV, or BPV. The vector incorporates the protein expression sequences. In some embodiments, the expression sequences are codon-optimized for expression in mammalian cells. In some embodiments, the vector also incorporates regulatory sequences including transcriptional activator binding sequences, transcriptional repressor binding sequences, enhancers, introns, and the like. In some embodiments, the viral vector supplies a constitutive or an inducible promoter. In some embodiments, the promoter is selected from EF1α, PGK1, MND, Ubc, CAG, CaMKIIa, and β-Actin promoter. In some embodiments, the promoter is selected from the SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the 0-interferon promoter, the hsp70 promoter and EF-1α promoter. In some embodiments, the promoter is an EF-1α promoter. In some embodiments, the promoter is an MND promoter.

In some embodiments, the viral vector supplies a transcription terminator or a polyadenylation signal. In some embodiments, the transcription terminator or polyadenylation signal is the BGH transcription terminator and polyadenylation signal.

In some embodiments, the vector is a plasmid selected from a prokaryotic plasmid, a eukaryotic plasmid, and a shuttle plasmid.

In some embodiments, the expression vector comprises one or more selection marker. In some embodiments, the selection markers are antibiotic resistance genes or other negative selection markers. In some embodiments, the selection markers comprise proteins whose mRNA is transcribed together with the BCMA-targeting CAR mRNA and the polycistronic transcript is cleaved prior to translation.

In some embodiments, the expression vector comprises polyadenylation sites. In some embodiments, the polyadenylation sites are SV-40 polyadenylation sites.

In some embodiments, the coding sequence of the BCMA-targeting CAR is introduced into the cells via a viral vector, such as e.g., AAV vector (AAV6) or any other suitable viral vector capable of delivering an adequate payload. In some embodiments, to facilitate homologous recombination, the coding sequence is joined to homology arms located 5′ (upstream or left) and 3′ (downstream or right) of the insertion site in the desired insertion site in the genome. In some embodiments, the homology arms are about 500 bp long. In some embodiments, the sequence coding for the BCMA-targeting CAR together with the homology arms are cloned into a viral vector plasmid. The plasmid is used to package the sequences into a virus.

In some embodiments, the cell (T cell, a natural killer (NK) cell, or an iNK cell) is contacted with a viral vector so that the genetic material delivered by the vector is integrated into the genome of the target cell and then expressed in the cell or on the cell surface. Transduced and transfected cells can be tested for transgene expression using methods well known in the art such as fluorescence-activated cell sorting (FACS), microfluidics-based screening, ELISA, or Western blot.

In some embodiments, the coding sequence for the BCMA-targeting CAR is introduced into the cell (T cell, a natural killer (NK) cell, or an iNK cell) as “naked” nucleic acid by electroporation as described e.g., in U.S. Pat. No. 6,410,319.

In some embodiments, an engineered CRISPR system is introduced into the cell (T cell, a natural killer (NK) cell, or an iNK cell). In some embodiments, the CRISPR system comprises a nucleic acid-guided endonuclease and nucleic acid-targeting nucleic acid (NATNA) guides (e.g., a CRISPR guide RNAs selected from tracrRNA, crRNA or a single guide RNA incorporating the elements of the tracrRNA and crRNA in a single molecule).

In some embodiments, NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, NATNA is an engineered guide RNA comprising one or more DNA residues (CRISPR hybrid RDNA or chRDNA). In some embodiments, NATNA is selected from the embodiments described in U.S. Pat. No. 9,650,617. Briefly, some chRDNA for use with a Type II CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of and a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, the components of the CRISPR system are introduced into the cell in the form of nucleic acids. In some embodiments, the components of the CRISPR system are introduced into the cell in the form of DNA coding for the nucleic acid-guided endonuclease and NATNA guides. In some embodiments, the gene coding for the nucleic acid-guided endonuclease (e.g., a CRISPR nuclease selected from Cas9 and Cas12a) is inserted into a plasmid capable of propagating in the cell. In some embodiments, the gene coding for the NATNA guides is inserted into a plasmid capable of propagating in the cell.

In some embodiments, the components of the CRISPR system, i.e., the nucleic acid-guided endonuclease and NATNA guides are introduced into the cell in the form of RNA, e.g., the mRNA coding for the nucleic acid-guided endonuclease along with the NATNA guides.

In some embodiments, the components of the CRISPR system, i.e., the nucleic acid-guided endonuclease and the NATNA guides are introduced into the cell as a preassembled nucleoprotein complex. In some embodiments, the components of the CRISPR system, i.e., the nucleic acid-guided endonuclease and the NATNA guides are introduced into the cell via any combination of different means, e.g., the endonuclease is introduced as the DNA via a plasmid containing the gene encoding the endonuclease while the guides are introduced in its final format as RNA (or RNA containing DNA nucleotides).

In some embodiments, the components of the CRISPR system, i.e., the nucleic acids encoding the nucleic acid-guided endonuclease and NATNA guides are introduced into the cell via electroporation.

In some embodiments, the components of the CRISPR system, i.e., the nucleic acids coding for the nucleic acid-guided endonuclease are introduced into the cell in the form of mRNA as described e.g., in the U.S. Pat. No. 10,584,352 via electroporation of viral pseudo-transduction as described therein.

In some embodiments, the coding sequence for the BCMA-targeting CAR is inserted into a double-strand break in the genome of the cell (T cell, a natural killer (NK) cell, or an iNK cell). In some embodiments, the introduction of the coding sequence coincides with inactivation of another gene by the insertion of the CAR gene (gene knock-out and simultaneous gene knock-in). In some embodiments, the insertion site and an inactivated gene is TRAC, CBLB, PDCD1, CTLA-4, LAG3, Tim3, BTLA, BY55, TIGIT, B7H5, LAIR1, SIGLEC10, and 2B4. In some embodiments, the BCMA-targeting CAR sequence is inserted into the T cell receptor alpha (TRAC) gene.

In some embodiments, prior to administration to a patient, the anti-BCMA CAR-T cells are assessed for their activity against B cells. In some embodiments, the anti-BCMA CAR-T cells are assessed for their activity against B cells derived from patients diagnosed with autoimmune disease.

In some embodiments, the activity of the anti-BCMA CAR-T cells against B cells is assessed in vitro as cytotoxicity against B cells derived from patients diagnosed with autoimmune disease.

In some embodiments, the in vitro assessment of cytotoxic properties of anti-BCMA CAR-T cells utilizes target cells or target cell lines. In some embodiments, the target cells are primary cells obtained from human blood samples. In some embodiments, the human samples are from patients diagnosed with autoimmune disease. In some embodiments, the human samples are control samples obtained from subjects free from autoimmune disease. In some embodiments, the samples are processed to extract blood fractions such as peripheral blood mononuclear cells (PBMCs), B cells or non-B cells. In some embodiments, the B cells are identified as cells expressing CD19 and/or CD20.

In some embodiments, target cells are established lymphoid cell lines. In some embodiments, target cells are established B cell lines. In some embodiments, target cells are established lymphoid tumor cell lines of B cell tumor cell lines.

In some embodiments, expression of BCMA in target cells is confirmed prior to assessing cytotoxicity of the anti-BCMA CAR-T cells. In some embodiments, expression of BCMA is confirmed by a method selected from flow cytometry with anti-BCMA antibody, staining with a label-conjugated anti-BCMA antibody, fluorescent in situ hybridization, Western blot or any other method known in the art to detect expression of a protein on the cell surface.

In some embodiments, cytotoxicity of the anti-BCMA CAR-T cells is assessed as lysis of B cells in vitro. The B cell lysis may be assessed by co-culturing the anti-BCMA CAR-T cells (effector cells or effectors) with a cell population comprising B cells or consisting of B cells. The co-culture may be established at different effector:target ratios (E:T ratios). In some embodiments, the E:T ratios are in the range of about 0.1:1 (1:10) to about 10:1. In some embodiments, two or more E:T ratios in the selected range are evaluated. In some embodiments, two or more or all of the E:T ratios selected from 0.125:1 (1:8), 0.25:1 (1:4), 0.5:1 (1:2), 1:1, 2:1, 4:1, 8:1 are evaluated.

In some embodiments, cell lysis is detected by labeling target cells with cell permeant stable fluorescent dyes (e.g., CellTrace™ Violet (CTV), ThermoFisher Scientific, Carlsbad, Cal.) in conjunction with viability dyes to measure specific lysis by flow cytometry. Cytotoxicity can also be determined by utilizing target cells expressing luciferase in cocultures with effector cells and measuring bioluminescence. Time lapse imaging can also be used to determine cell lysis by either incorporating a viability dye and measuring increase in fluorescence or by utilizing cells containing a fluorescent reporter and measuring decrease in fluorescence. Impedance-based systems like the Agilent xCELLigence system can also provide dynamic real time monitoring of cell lysis.

In some embodiments, a control experiment is performed assessing lysis of cell populations consisting of non-B cells by the anti-BCMA CAR-T cells. In some embodiments, a control experiment is performed assessing lysis of cell populations comprising both B cells and non-B cells (e.g., PBMCs) by the anti-BCMA CAR-T cells.

In some embodiments, B cell lysis by the anti-BCMA CAR-T cells is compared in primary cell samples from autoimmune patients and primary cell samples from subjects free from autoimmune disease.

In some embodiments, the anti-BCMA CAR-T cell population effecting the highest percentage of B cell lysis is selected for administration to a patient suffering from autoimmune disease. In some embodiments, the anti-BCMA CAR-T cell population effecting a high percentage of B cell lysis but having low non-B cell lysis is selected for administration to a patient suffering from autoimmune disease.

In some embodiments, the activity of the anti-BCMA CAR-T cells against B cells is assessed in vitro as decrease in autoantibody secretion by the B cells. In some embodiments, the autoantibody is anti-DNA IgG. In some embodiments, autoantibody secretion is assessed by co-culturing anti-BCMA CAR-T cell (effectors, E) with a cell population comprising B cells (targets, T). In some embodiments, the co-culture is at E:T ratio in the range of about 1:10 to about 10:1. In some embodiments, the co-culture is at E:T ratio of about 1:1. In some embodiments, the autoantibodies in the co-culture supernatant are assessed qualitatively or quantitatively. The autoantibodies can be assessed as total IgG in the supernatant. Specific species of autoantibodies (e.g., anti-dsDNA IgG characteristic of SLE) can be detected with an antibody-based or antibody conjugate-based assay such as Western blotting or ELISA and similar secondary antibody-based methods with colorimetric, chemiluminescent, or fluorescent detection methods. Anti-dsDNA antibodies, can also be detected using Farr radioimmunoassay, which measures radiolabeled dsDNA bound to anti-dsDNA antibodies, or using Crithidia luciliae indirect immunofluorescence test (CLIFT).

In some embodiments, the invention comprises compositions including cells (T cells, natural killer (NK) cells, or iNK cells) expressing a BCMA-targeting protein. In some embodiments, the composition comprises cytotoxic CAR-T cells or CAR-NK cells expressing an anti-BCMA chimeric antigen receptor (CAR). In some embodiments, the compositions include the cells, and one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

In some embodiments, the composition further comprises an antimicrobial agent for preventing or deterring microbial growth. In some embodiments, the antimicrobial agent is selected from benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

In some embodiments, the composition further comprises an antioxidant added to prevent the deterioration of the lymphocytes. In some embodiments, the antioxidant is selected from ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some embodiments, the composition further comprises a surfactant. In some embodiments, the surfactant is selected from polysorbates, sorbitan esters, lipids, such as phospholipids (lecithin and other phosphatidylcholines), phosphatidylethanolamines, fatty acids and fatty esters; steroids, such as cholesterol.

In some embodiments, the composition further comprises a freezing agent such as 3% to 12% dimethylsulfoxide (DMSO) or 1% to 5% human albumin.

The number of adoptive cells, such as T cells, NK cells, CAR-T cells or CAR-NK cells, in the composition will vary depending on a number of factors but will optimally be a therapeutically effective dose per vial.

A minimum or optimal therapeutically effective dose can be determined experimentally by repeated administration of increasing amounts of the composition in order to determine which amount produces a reduction in symptoms of autoimmune disease.

A maximum or optimal therapeutically effective dose can be determined experimentally by repeated administration of decreasing amounts of the composition in order to determine which amount produces a reduction in symptoms of autoimmune disease while not producing undesirable side effects or producing an acceptable degree of undesirable side effects.

The invention includes a step of administering to the patient a composition comprising immune cells (T cells, NK cells or iNK cells) expressing a BCMA-targeting protein.

In some embodiments, prior to administration of the immune cells, the patient undergoes a lymphodepletion pre-treatment to reduce any immune system attack against the administered immune cells.

In some embodiments, the patient is pre-treated with an immunosuppressor known to be safe and effective against autoimmune disease, see e.g., Fava A., and Petri, M. (2019) Systemic lupus erythematosus: diagnosis and clinical management, J. Autoimmun. 96:1-13.

In some embodiments, the immunosuppressor is cyclophosphamide, an alkylating agent with a history of use in lupus patients and known to deplete T and B cells.

In some embodiments, the immunosuppressor is azathioprine, a purine analogue with a history of use in lupus patients.

In some embodiments, the immunosuppressor is methotrexate, an antimetabolite with a history of use in lupus patients and known to suppress proinflammatory T cells.

In some embodiments, the immunosuppressor is mycophenolate, an agent depleting guanoside nucleotides and having a history of use in lupus patients and known to inhibit proliferation of T and B cells.

In some embodiments, the immunosuppressor is a calcineurin inhibitor (e.g., volcosporin) with a history of use in lupus patients and known to reduce T cell activity.

In some embodiments, the anti-BCMA CAR-T cells or anti-BCMA CAR-NK cells possess an armoring modification described herein. In case of armored cells, the lymphodepletion regimen is milder, i.e., uses lower doses of lymphodepleting agents compared to the lymphodepletion regimen used for non-armored CAR-T cells or CAR-NK cells. In some embodiments, the lymphodepletion includes administration of cyclophosphamide at a dose lower than 60 mg/kg per day for 2 days and administration of fludarabine at a dose lower than 25 mg/m² per day for 5 days.

In some embodiments, the lymphodepletion includes administration of cyclophosphamide at a dose 300 mg/m² per day for 3 days and administration of fludarabine at a dose 30 mg/m² per day for 3 days. In some embodiments, the lymphodepletion includes simultaneous administration of cyclophosphamide and fludarabine. In some embodiments, the lymphodepletion includes sequential administration of cyclophosphamide and fludarabine.

In some embodiments, lower doses of lymphodepletion agents are employed. In some embodiments, a lower dose is used if the standard dose is not tolerated by the autoimmune disease patient. In some embodiments, a lower dose is used if a high rate of disease modification is observed with the standard dose.

At the end of the lymphodepletion pre-treatment, the patient is administered a composition including no greater than 50,000,000 (equivalent to no greater than 8×10⁴/kg) of immune cells expressing an anti-BCMA protein. In some embodiments, the patient is administered 50,000 (equivalent to 800/kg) of anti-BCMA allogeneic CAR-T cells.

The dose of BCMA targeting cells (such as anti-BCMA CAR-T cells and CAR-NK cells) cells needed to treat autoimmune disease is substantially lower than the dose of the CAR-T or CAR-NK cells needed to treat tumors. In addition, the dose of allogeneic CAR-T or CAR-NK cells needed to achieve a therapeutic effect on tumors can be lower than the dose of autologous CAR-T or CAR-NK cells. Table 1 lists the doses of autologous anti-BCMA CAR-T cell therapies ABECMA® and CARVYKTI® compared to an experimental allogeneic anti-BCMA CAR-T cell treatment CB-011 (see Garner, E., Degagne, E., et al., A BCMA-specific allogeneic CAR-T cell therapy (CB-011) genome engineered to express an HLA-E fusion transgene to prevent immune cell rejection, Poster LB009, American Association for Cancer Research (AACR) Annual Meeting, Apr. 10, 2022).

TABLE 1
Dose comparison among allogeneic and autologous
CAR-T cell treatments for oncology indications

| | | | | Dose compared |
|---|---|---|---|---|
| Therapy | Type | Dose | | to CB-011 |
| CB-011 | allogeneic | 5 × 10⁷ | cells | n/a |
| ABECMA ® | autologous | 3-4.6 × 10⁸ | cells | 6-9 × greater |
| CARVYKTI ® | autologous | 5 × 10⁷ | cells | Same |

(Camelid
antibody)

In some embodiments, the dose of anti-BCMA CAR-T cells or CAR-NK cells for a human patient is about 0.1% ( 1/1000^{th}) of the dose of anti-BCMA CAR-expressing cells compared to the dose of the same CAR-T cells administered to treat tumors. For example, for CB-011 allogeneic anti-BCMA CAR-T cells, the dose is between 5×10⁴ (50,000) and 5×10⁸ (500,000,000) of CAR-T cells or CAR-NK cells compared to 5×10⁷ (50,000,000) of CAR-T cells or CAR-NK cells used to treat multiple myeloma. Expressed in cells per kilogram of body weight, the dose is between 8×10² (800) cells/kg and 8×10⁶ (8,000,000) cells/kg of allogeneic CAR-T (or CAR-NK) compared to 8×10⁵ CAR-T cells/kg used to treat multiple myeloma.

In some embodiments, the patient is administered fewer than 500,000,000 (equivalent to fewer than 8×10⁶/kg) of allogeneic anti-BCMA CAR-expressing cells.

In some embodiments, the patient is administered at least 50,000 (equivalent to at least 800/kg) of allogeneic anti-BCMA CAR-expressing cells.

In some embodiments, the invention comprises administering to the patient the anti-BCMA allogeneic CAR-T cells or CAR-NK cells at a frequency of 2-4 times per year.

In some embodiments the patient is treated with anti-BCMA allogeneic CAR-T cells more or less frequently than 2-4 times per year based on the symptom assessment described herein including blood and urine analysis, and visual assessment to detect the progress of treatment or progression of the disease and any side effects.

In some embodiments, the therapeutic composition is administered to a patient by a route selected from intravenous, parenteral, intrathecal, local, and intramuscular. In some embodiments the administration is by infusion and the infusion is selected from a single sustained dose, a prolonged continuous infusion, and multiple infusions.

### EXAMPLES

### Example 1. CB-011: Allogeneic Anti-BCMA CAR-T Cells

The allogeneic anti-BCMA CAR-T cells with armoring referred to as CB-011 were developed for the treatment of multiple myeloma. (See Garner, E., Degagne, E., et al., A BCMA-specific allogeneic CAR-T cell therapy (CB-011) genome engineered to express an HLA-E fusion transgene to prevent immune cell rejection, Poster LB009, American Association for Cancer Research (AACR) Annual Meeting, Apr. 10, 2022.)

Briefly, the CB-011 cells were generated from T cells obtained by leukapheresis of healthy donor blood samples. CRISPR Cas12a endonuclease with chRDNAs (CRISPR hybrid RNA-DNA guides) was used for genome editing. The anti-BCMA CAR transgene (FIG. 1) was delivered via an AAV vector and inserted into the T cell receptor alpha chain (TRAC) locus on chromosome 14. Additionally, a fusion of the beta-2 microglobulin (B2M) and HLA-E sequences was inserted into the B2M locus using Cas12a/chRDNAs resulting in abrogation of B2M expression and expression of the B2M-HLA-E-peptide fusion on the cell surface.

### Example 2. (Prophetic) Specific Lysis of B Cells by the Anti-BCMA CAR-T Cells (CB-011)

In this example, the anti-BCMA CAR-T cells (Example 1) are cocultured with cellular fractions obtained from patients suffering from autoimmune disease and with control samples from autoimmune disease-free individuals. Depending on the nature of the autoimmune disease, patient B cells may be obtained from blood plasma or bone marrow (lupus or other indications), cerebrospinal fluid (MS), or synovial fluid (RA). As a control, donor-matched T cells with inactivated TRAC locus but no CAR insertion (TRAC KO) are used. The target cells are labeled with CTV to distinguish them from effector cells. Non-diseased B cell controls are cocultured with CB-011 at the following E:T ratios: 8:1, 4:1, 2:1, 1:1, 0.5:1 0.25:1, 0.125:1, 0:1. Autoimmune patient-derived cellular fractions are cocultured at the following E:T ratios: 0.5:1, 0.25:1, 0.125:1, 0.0625:1, 0.03125:1 0.015625:1, 0.0078125:1, 0:1. Cocultures are maintained for 24 hours, after which cocultures are stained with a B cell marker-specific antibody (such as CD19 or CD20) and with a viability dye (such as propidium iodide (PI)) for cytotoxicity measurement through flow cytometry (iQue Screener Plus, Intellicyt, Albuquerque, N.M.). Cytotoxicity is determined by gating on the live cell population within the CTV-labeled target cell populations, or within B cell and non-B cell populations of the CTV-labeled target cells. Specific lysis is calculated using the following equation for each well: Specific lysis=1−(% of live target cells in coculture sample/% of live target cells in target-only sample). Specific lysis curves are then generated for different samples, and area under the curve (AUC) measurements of specific lysis is determined for the different populations and conditions.

### Example 3. (Prophetic) Decrease in Autoantibody Secretion by B Cells in the Presence of the Anti-BCMA CAR-T Cells (CB-011)

In this example, the CB-011 allogeneic anti-BCMA CAR-T cells (Example 1) are cocultured with cellular fractions obtained from patients suffering from autoimmune disease and with control samples from autoimmune disease-free individuals. Depending on the nature of the autoimmune disease, patient B cells may be obtained from blood plasma or bone marrow (lupus or other indications), cerebrospinal fluid (MS), or synovial fluid (RA). As controls, target cells are also cultured alone or co-cultured with donor-matched T cells with inactivated TRAC locus but no CAR insertion (TRAC KO). Non-diseased B cell controls are co-cultured with effector cells at a 1:1 E:T ratio, and autoimmune-derived cellular fractions are co-cultured with effector cells at a 1:4 E:T ratio to account for B cells being only a fraction of the PBMCs. Co-cultures are maintained for 6 days in the presence of ODN2006, a CpG oligonucleotide that strongly activates B cells through TLR9 activation. After 6 days, supernatants are harvested from the cocultures. Total IgG and anti-dsDNA IgG concentration are measured in the co-culture supernatants using ELISA kits specific for total IgG detection (Invitrogen, Carlsbad, Cal.) or anti-dsDNA IgG detection (Abnova, Taipei City, Taiwan). The measurements of autoimmune antibody concentrations in co-cultures of CB-011 with SLE-derived cellular fractions and RA-derived cellular fractions are compared.

### Example 4. (Prophetic) Administering the Anti-BCMA Allogeneic CAR-T Cells to Measurably Alleviate the Symptoms of Lupus

In this example, a human patient is subjected to one or more of urine analysis, blood analysis (including total blood count), physical assessment and is diagnosed with lupus if one or more of the following is present: proteinuria, alopecia, organ enlargement, the presence of hypercellular glomeruli, IgG tissue deposits, IgM and IgG antibody titers and IgG or IgM antinuclear antibody in blood serum, an increase in the total number or concentration of B cells in the blood plasma, and the presence of skin lesions or discoloration.

The patient undergoes lymphodepletion pre-treatment consisting of cyclophosphamide at 300 mg/m² per day for 3 days and fludarabine at 30 mg/m² per day for 3 days, either simultaneously or consecutively.

At the end of the lymphodepletion pre-treatment, the patient is administered a composition including minimally 50,000 (equivalent to 800/kg) of CB-011 anti-BCMA allogeneic CAR-T cells.

Starting with one week post-administration the patient is assessed by one or more of urine analysis, blood analysis (including total blood count), and physical evaluation to detect any diminution of previously existing symptoms of lupus selected from proteinuria, alopecia, organ enlargement, the presence of hypercellular glomeruli, IgG tissue deposits, IgM and IgG antibody titers and IgG or IgM antinuclear antibody in blood serum, an increase in the total number or concentration of CD3⁺CD8⁺ cells in the blood plasma, an increase in the total number or concentration of B cells in the blood plasma, complement C3 levels, complement C4 levels, and the presence of skin lesions or discoloration.

The total number or concentration of B cells in the blood plasma is assessed by flow cytometry. The IgG or IgM antinuclear antibody in blood serum is assessed by ELISA.

The levels of the major complement proteins are assessed with a commercial test, e.g., a functional hemolytic test CH50 or CH100.

The patient is also assessed for presence (persistence) of the CB-011 anti-BCMA allogeneic CAR-T cells. These cells are detected by flow cytometry, ELISA, fluorescent microscopy, fluorescent in situ hybridization (FISH), PCR, ddPCR, or RT-PCR aimed at detecting the presence of the BCMA-targeting CAR, the gene encoding the CAR, or the mRNA encoding the CAR.

If no diminution of the symptoms is observed, the patient is administered another dose or a greater dose of the CB-011 anti-BCMA allogeneic CAR-T cells.

If a low number or none of the CB-011 anti-BCMA allogeneic CAR-T cells are detected in the patient's circulation, the patient is administered another dose or a greater dose of the CB-011 anti-BCMA allogeneic CAR-T cells.

Alternatively, if a low number or none of the CB-011 anti-BCMA allogeneic CAR-T cells are detected in the patient's circulation, the patient is administered another dose or a greater dose of the lymphodepletion regimen and another dose of the CB-011 anti-BCMA allogeneic CAR-T cells.

### Example 5. Specific Lysis of B Cells by Anti-BCMA CAR-T Cells

In this example, the anti-BCMA CAR-T cells were cocultured with cellular fractions obtained from blood samples of autoimmune patients or with isolated non-diseased B cells. As a control effector cell sample, donor-matched T cells with inactivated TRAC locus but no anti-BCMA CAR insertion (TRAC KO) were used. Briefly, targets were labeled with CTV to distinguish them from effector cells. Non-diseased B cells were cocultured at the following E:T ratios: 8:1, 4:1, 2:1, 1:1, 0.5:1 0.25:1, 0.125:1, 0:1. Autoimmune patient-derived cells were cocultured at the following E:T ratios: 0.5:1, 0.25:1, 0.125:1, 0.0625:1, 0.03125:1 0.015625:1, 0.0078125:1, 0:1. Cocultures were maintained for 24 hours, after which cocultures were stained with an antibody specific for a B cell marker (CD19 or CD20) and with a viability dye propidium iodide (PI) for cytotoxicity measurement through flow cytometry (iQue Screener Plus, Intellicyt, Albuquerque, N.M.). Cytotoxicity was determined by gating on the live cell population within the CTV-labeled target cell populations or within B cell and non-B cell populations of the CTV-labeled target cells. Specific lysis was calculated using the following equation for each well: Specific lysis=1−(% of live target cells in coculture sample/% of live target cells in target only sample). Specific lysis curves were then be generated for different samples, and area under the curve (AUC) measurements of specific lysis was calculated.

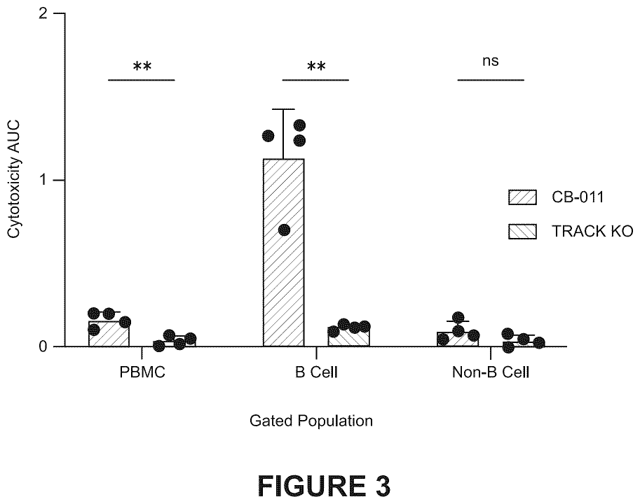

FIG. 3 shows results of in vitro cytotoxicity assessment of anti-BCMA CAR-T cells (CB-011, Example 1) in SLE-derived cellular fractions. Cytotoxicity is expressed as area under the curve (AUC) measurement of specific lysis of PBMCs, B-cells and non-B-cells from SLE patients by the CB-011 allogeneic anti-BCMA CAR-T cells. Data represents 4 independent donors (4 SLE patient-derived PBMCs). Error bars represent average ±SD. ns (not significant) indicates p>0.05 and ** indicates p≤0.01 by paired t-test between CB-010 and TRAC KO coculture conditions.

### Example 6. Decrease in Autoantibody Secretion by the SLE Cells in the Presence of Anti-BCMA Car-T Cells

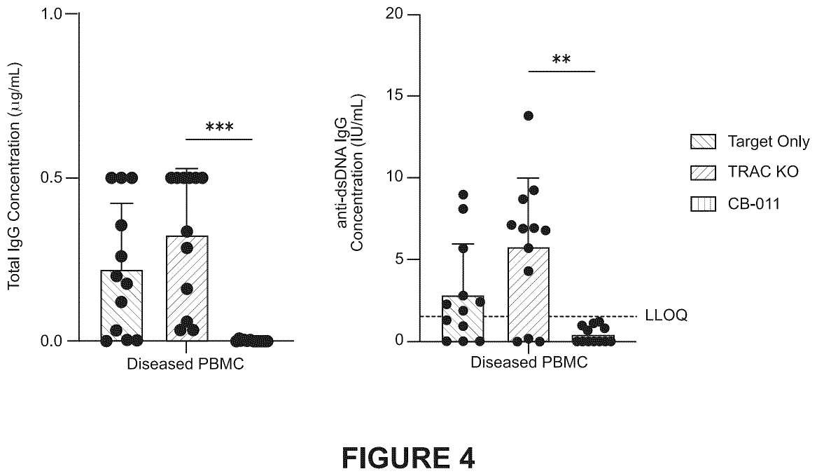

In this example, the CB-011 allogeneic anti-BCMA CAR-T cells (Example 1) were cocultured with cellular fractions obtained from blood samples of autoimmune patients or with isolated non-diseased B cells. As controls, targets were also cultured alone or cocultured with donor-matched T cells with inactivated TRAC locus but no anti-BCMA CAR insertion (TRAC KO). Non-diseased B cells were cocultured with effector cells at a 1:1 E:T ratio, and autoimmune-derived cellular fractions were cocultured with effector cells at a 1:4 E:T ratio to account for B cells being a fraction of the PBMCs. Cocultures were maintained for 7 days in the presence of ODN2006, a CpG oligonucleotide that strongly activates B cells through TLR9 activation. After 7 days, supernatants were harvested from the cocultures. Total IgG and anti-dsDNA IgG concentration were measured in the co-culture supernatants using ELISA kits specific for total IgG (Invitrogen) or anti-dsDNA IgG (Abnova) detection. Results are shown in FIG. 4 as measurements of total or autoimmune antibody concentrations in co-cultures of CB-011 with SLE-derived cellular fractions. Data represents 4 independent donors (4 SLE patient-derived PBMCs). Error bars represent average ±SD. ** indicates p≤0.01, *** indicates p≤0.001 by paired t-test between TRAC KO and CB-011 coculture conditions.

Anti-BCMA scFv
(SEQ ID NO: 1)
QVQLVQSGAEVKKPGSSVKVSCKASGYTFTSYVMHWVRQAPGQGLEWMG
YIIPYNDATKYNEKFKGRVTITADKSTSTAYMELSSLRSEDTAVYYCAR
YNYDGYFDVWGQGTLVTVSSGGGGSGGGGSGGGGSEILTQSPATLSLSP
GERATLSCRASQSISDYLHWQQKPGQAPRLLIYYASQSITGIPARFSGS
GSGTDFTLTISSLEPEDFAVYYCQNGHSFPPTFGGGTKVEIK

### LITERATURE

Dogan, et al., (2020) B-cell maturation antigen expression across hematologic cancers: a systematic literature review, Blood cancer Journal, 10:73.
Gornalusse et al., (2017) HLA-E-expressing pluripotent stem cells escape allogeneic responses and lysis by NK cells, Nat. Biotechnol. (2017) 35:765-772.
Zhang et al., (2021) Treatment of systemic lupus erythematosus using BCMA-CD19 compound CAR, Stem Cell Reviews and Reports, 17:2120-2123.
Kansal et al., (2019) Sustained B cell depletion by CD-19 targeted Car-T cells is a highly effective treatment for murine lupus, Science Trans. Med., 11 eeav1648.
Jin et al., (2021) Therapeutic efficacy of anti-CD19 CAR-T cells in a mouse model of systemic lupus erythematosus, Cellular and Molecular Immun., 18(6):1896-1903.
DiLillo et al., (2008) Maintenance of long-lived plasma cells and serological memory despite mature and memory B cell depletion during CD20 immunotherapy in mice, J Immunol., 180(1):361-371.
Khodadadi, et al., (2015) Bortezomib plus continuous B cell depletion results in sustained plasma cell depletion and amelioration of lupus nephritis in NZB/W F1 mice, PLOS One 10:e0135081.
Garner, E., Degagne, E., et al., A BCMA-specific allogeneic CAR-T cell therapy (CB-011) genome engineered to express an HLA-E fusion transgene to prevent immune cell rejection, Poster LB009, American Association for Cancer Research (AACR) Annual Meeting, Apr. 10, 2022.
Qin, et al., (2023) Anti-BCMA CAR-T cell therapy CT103A in relapsed or refractory AQP4-IgG seropositive neuromyelitis optica spectrum disorders: phase 1 trial interim results, Signal Transduction and Targeted Therapy, 8:5.
Gupta, et al., (2023) CAR-T cell-mediated B cell depletion in central nervous system autoimmunity, Neurology Neuroimmunology and Neuroinflammation, 10:e200080.
Ellebrecht, et al. (2016) Reengineering chimeric antigen receptor T cells for targeted therapy of autoimmune disease. Science 353:179-184.
Zhang, et al., (2021) In vitro elimination of autoreactive B cells from rheumatoid arthritis patients by universal chimeric antigen receptor T cells. Ann Rheum Dis.; 80:176-184.

While the invention has been described in detail with reference to specific examples, it will be apparent to one skilled in the art that various modifications can be made within the scope of this invention. Thus, the scope of the invention should not be limited by the examples described herein, but by the claims presented below.

What is claimed is:

1.-52. (canceled)

53. A method of treating an autoimmune disease in a patient, comprising:
administering to the patient an amount of a composition comprising allogenic B-cell maturation antigen (BCMA)-targeting engineered chimeric antigen receptor (CAR)-T cells expressing an anti-BCMA chimeric antigen receptor (CAR), wherein the CAR comprises a single-chain variable fragment (scFv) comprising SEQ ID NO: 1.

54. The method of claim 53, wherein the allogeneic BCMA-targeting engineered CAR-T cells further comprise a disruption of the beta-2-microglobulin (B2M) gene and an insertion of a beta-2-microglobulin-human-leukocyte-antigen-E (B2M-HLA-E) peptide into the B2M gene.

55. The method of claim 54 wherein the anti-BCMA CAR further comprises a CD8 hinge, a CD8 transmembrane domain, a 4-1BB co-stimulatory domain, and a CD3 zeta signaling domain.

56. The method of claim 55, wherein the autoimmune disease is selected from the group consisting of Systemic Lupus Erythematosus (SLE), Rheumatoid Arthritis (RA), Type 1 Diabetes (T1D), Sjögren's syndrome, Neuromyelitis optica syndrome disorders (NMOSD), Myasthenia Gravis, Ankylosing spondylitis, Pemphigus vulgaris (PV), and Multiple Sclerosis (MS).

57. The method of claim 55, wherein the anti-BCMA CAR is encoded by a nucleic acid comprising a coding sequence for the anti-BCMA CAR and a promoter, and wherein the nucleic acid is integrated into the genome of the CAR-T cells.

58. The method of claim 57, wherein prior to integration the nucleic acid is delivered into the CAR-T cells via a viral vector.

59. The method of claim 57, wherein the integration of the nucleic acid is performed using a clustered regularly interspaced short palindromic repeats (CRISPR) nuclease and a nucleic acid-targeting nucleic acid (NATNA).

60. The method of claim 59, wherein the CRISPR nuclease comprises a Cas12a endonuclease.

61. The method of claim 60, wherein the NATNA comprises RNA nucleotides.

62. The method of claim 61, wherein the NATNA further comprises DNA nucleotides.

63. The method of claim 55, wherein the amount of the composition administered to the patient comprises between 50,000 and 500,000,000 of the BCMA-targeting engineered CAR-T cells.

64. The method of claim 55, wherein the amount of the composition administered to the patient comprises between 10,000 and 100,000,000 of the BCMA-targeting engineered CAR-T cells.

65. The method of claim 55, wherein the administering of the composition to the patient is performed intravenously.

66. The method of claim 55, wherein prior to the administering of the composition to the patient, the patient undergoes lymphodepletion comprising administration of a compound selected from the group consisting of cyclophosphamide, fludarabine, azathioprine, methotrexate, mycophenolate, a calcineurin inhibitor, and volcosporin.

67. The method of claim 66, wherein the lymphodepletion comprises cyclophosphamide at 300 mg/m² per day for three days prior to the administering of the composition to the patient.

68. The method of claim 67, wherein the lymphodepletion further comprises fludarabine at 30 mg/m² per day for three days prior to the administering of the composition to the patient.

69. The method of claim 55, wherein the composition further comprises one or more pharmaceutically acceptable excipients.

70. The method of claim 69, wherein the one or more pharmaceutically acceptable excipients are selected from the group consisting of carbohydrates, inorganic salts, antimicrobial agents, antioxidants, surfactants, buffers, acids, bases, and combinations thereof.

71. The method of claim 55, wherein the composition further comprises a freezing agent.

72. The method of claim 71, wherein the freezing agent is selected from the group consisting of 3% to 12% dimethylsulfoxide (DMSO) and 1% to 5% human albumin.


SEQUENCE LISTING (1 sequence)

SEQ ID NO: 1 - protein, 238 aa, synthetic construct
  REGION 1..238: note = Anti-BCMA scFv
QVQLVQSGAEVKKPGSSVKVSCKASGYTFTSYVMHWVRQAPGQGLEWMGYIIPYNDATKYNEKFKGRVTITADKSTSTAYMELSSLRSEDTAVYYCARYNYDGYFDVWGQGTLVTVSSGGGGSGGGGSGGGGSEILTQSPATLSLSPGERATLSCRASQSISDYLHWQQKPGQAPRLLIYYASQSITGIPARFSGSGSGTDFTLTISSLEPEDFAVYYCQNGHSFPPTFGGGTKVEIK